(19) United States
(12) Patent Application Publication
(10) Pub. No.: US 2006/0106420 A1
(43) Pub. Date: May 18, 2006

(54) Patch for treating a septal defect

(75) Inventors: Mark J. Dolan, Santa Rosa, CA (US); Asha S. Nayak, Menlo Park, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 10/988,462

(22) Filed: Nov. 12, 2004

(51) Int. Cl. A61B 17/08

(52) U.S. Cl. 606/213

(57) ABSTRACT

A system for treating a septal defect comprises a treatment device slidably received within a catheter. The device includes a patch attached to a support. The support has a body segment and a plurality of leg segments that self-expand radially outward as the device is released from the catheter. A method for treating a septal defect comprises delivering the treatment device in the catheter proximate a septal defect. The device is slid in a distal direction to release a portion of each leg segment from the catheter, the leg segments partially expanded radially outward. The leg segments are placed in contact with tissue surrounding the septal defect. The device is slid farther until the leg segments are fully released from the catheter and fully expanded, thereby implanting a distal portion of each leg segment in tissue surrounding the septal defect and positioning the patch against the septal defect.

### TECHNICAL FIELD

This invention relates generally to medical devices and particularly to a device, system, and method for treating a septal defect such as a patent foramen ovale.

### BACKGROUND OF THE INVENTION

Fetuses have a normal opening, the foramen ovale, between the left and right atria of the heart. This opening allows blood to bypass the lungs while a child is in utero. The opening normally closes soon after a child is born and pulmonary circulation is established.

In some individuals, the foramen ovale fails to close (i.e., remains patent), resulting in a condition called patent foramen ovale (PFO). Many individuals with PFO experience no symptoms. However, PFO can lead to strokes when small, often undetectable, clots form in the pelvis or lower extremities. If a clot breaks loose, it can travel through veins to the heart and pass through the patent foramen ovale to the left (arterial) side of the heart. The clot may then travel with the arterial blood to the brain and become lodged there, preventing blood flow to a part of the brain, resulting in a stroke.

Other atrial and ventricular septal defects can occur and are commonly called “holes” in the heart. Most of these defects are congenital, but defects can occur rarely as a serious complication of a heart attack.

Septal defects may be repaired surgically. Although relatively simple, surgical therapy is invasive, costly, and painful, and is associated with all the usual risks of cardiac surgery.

Catheter-based treatment is also possible. In the case of PFO, treatment may involve stapling the foramen ovale closed. This method of treatment requires flaps of tissue that overlap sufficiently to effect closure of an opening when the flaps are stapled together. While flaps are usually present in PFO, the foramen ovale typically being a tunnel with openings that are not opposite each other but instead are displaced longitudinally, the longitudinal displacement may not be adequate to allow a staple device to pass through both flaps simultaneously, resulting in the staple passing through only the nearest flap and not engaging the second flap.

Even where the overlap is adequate for stapling, a certain amount of force may be required to ensure that a device passes through both flaps, and the device may need to be relatively long and/or remain in a fully open or straight position for some time before engaging the second flap. For best closure, the staple may need to pass entirely through both flaps, thus extending into the left atrium, which may pose a risk of embolus formation.

Another disadvantage of such systems is that the staples are typically ejected from a delivery catheter rather than having a controlled delivery. If the opening is inadequately closed by the staple, using a second staple may not be possible or desirable, resulting in the need for surgical closure of the PFO.

Therefore, it would be desirable to have a device, system, and method for treating a septal defect that overcomes the aforementioned and other disadvantages.

### SUMMARY OF THE INVENTION

One aspect according to the present invention is a device for treating a septal defect, comprising a support and a patch attached to the support. The support includes a body segment and a plurality of flexible leg segments. The leg segments self-expand radially outward from the body segment as the support is released from a delivery catheter.

Another aspect according to the present invention is a system for treating a septal defect comprising a delivery catheter and a treatment device slidably received within a lumen of the delivery catheter. The treatment device has a support and a patch attached to the support. The support includes a body segment and a plurality of flexible leg segments. The leg segments self-expand radially outward from the body segment as the device is released from the delivery catheter.

Yet another aspect according to the present invention is an indwelling medical system comprising an elongated member, an anchor, and a retractable sheath. A portion of the elongated member is encircled by the anchor, which includes a body segment and a plurality of flexible leg segments. The retractable sheath encloses the flexible leg segments. At least a plurality of the flexible leg segments self-expand radially outward from the body segment when the sheath is retracted.

Still another aspect according to the present invention is a method of treating a septal defect. A treatment device is delivered in a lumen of a catheter proximate a septal defect. The treatment device has a support and a patch attached to the support. The support includes a body segment and a plurality of flexible leg segments. The treatment device is slid in a distal direction such that a portion of each leg segment is released from the distal end of the catheter and the leg segments are partially expanded radially outward from the body segment. As used herein, the terms “distal” and “proximal” are with reference to the treating clinician during deployment of the device. The treatment device is positioned such that the leg segments contact tissue surrounding the septal defect. The treatment device is again slid in a distal direction such that each leg segment is fully released from the distal end of the catheter and the leg segments are fully expanded radially outward from the body segment. In response to the full radial expansion of the leg segments, a distal portion of each leg segment is implanted in the tissue surrounding the septal defect, thereby positioning the patch against the septal defect.

The aforementioned and other features and advantages of the invention will become further apparent from the following detailed description, read in conjunction with the accompanying drawings, which are not to scale.

### BRIEF DESCRIPTION OF THE DRAWINGS

FIG. 1 is an isometric view of one embodiment of a device for treating a septal defect, in accordance with the present invention;

FIG. 2-4 are isometric views of one embodiment of a system for treating a septal defect, in accordance with the present invention, showing a progression of deployment of a treatment device in accordance with the present invention, the treatment device being shown within a delivery catheter in cross section;

FIG. 5 is a schematic view illustrating placement of a treatment device proximate a septal defect, in accordance with the present invention; and

FIG. 6 is a schematic view illustrating one embodiment of an indwelling medical system, in accordance with the present invention;

FIG. 7 is a schematic view illustrating another embodiment of an indwelling medical system, in accordance with the present invention; and

FIG. 8 is a flow diagram of one embodiment of a method of treating a septal defect, in accordance with the present invention.

### DETAILED DESCRIPTION

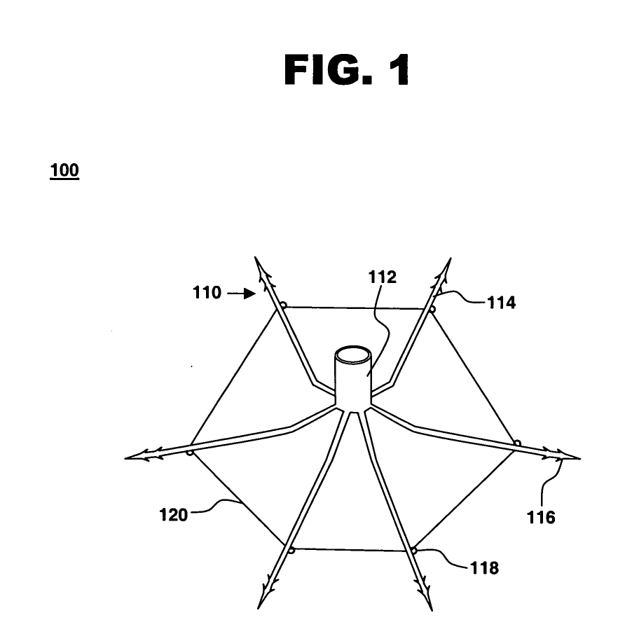

One aspect according to the present invention is a device for treating a septal defect. One embodiment of the device, in accordance with the present invention, is illustrated in FIG. 1 at 100. Treatment device 100 comprises a support 110 and a patch 120. Support 110 includes a body segment 112 and a plurality of leg segments 114. The leg segments include barbs 116 and patch attachment structures 118.

In the present embodiment, support 110 comprises a section of tubing having evenly spaced longitudinal slots cut into an end portion of the tubing to form body segment 112 and six flexible leg segments 114. One skilled in the art will appreciate that the number and shape of the leg segments may be varied. The slots may be, for example, rectangular, v-shaped, u-shaped, or Ω-shaped (omega-shaped). In another embodiment, support 110 may be manufactured by cutting, stamping, or otherwise forming the device from material not previously shaped into a tube. In either embodiment, the leg portions may be formed separately from the body portion of the contracting device and assembled to create an integral whole.

Support 110 is manufactured using one or more materials. At least leg segments 114 of support 110 comprise a material capable of being preset into a desired shape, for example that shown in FIG. 1. Such materials include, but are not limited to, a nickel-titanium alloy, a nickel-cobalt alloy, another cobalt alloy, a thermoset plastic, stainless steel, a stainless steel alloy, a biocompatible shape-memory material, a bioabsorbable shape-memory material, a biocompatible superelastic material, a bioabsorbable superelastic material, combinations thereof, and the like. An antithrombotic component may be included in the chemical composition of a polymer used to form the device. Alternatively, a polymeric or metallic device may be coated with a polymer that releases an anticoagulant and thereby reduces the risk of thrombus formation. If desired, additional therapeutic agents or combinations of agents may be used, including antibiotics and anti-inflammatories.

During manufacture, leg segments 114 are bent outward and heat set or otherwise set such that each of the leg segments is self-expanding radially outward at an angle of between 60 and 100 degrees from the longitudinal axis of body portion 112 when the treatment device is released from a delivery catheter. Leg segments 114 are in a radially compressed, folded configuration while device 100 is within the delivery catheter. As support 110 is released from the delivery catheter, leg segments 114 resume their preset shape, self-expanding radially outward from body segment 112. When the device is released adjacent to a septal defect, this radial expansion along with the force keeping the delivery catheter adjacent the septal defect implants a distal portion of each leg segment into tissue surrounding a septal defect. The leg segments are preferably implanted between the surfaces of the septal wall but may also pass through the tissue, piercing the septal wall.

To increase the ability of leg segments 114 to grip the tissue surrounding a septal defect, one or more barbs 116 may be formed on or adjacent to the distal tip of each leg segment as seen in FIG. 1. One skilled in the art will recognize that other shapes and orientations of barbs may be used to secure leg segments 114 within the tissue.

Each leg segment 114 may include an attachment structure 118 for attaching patch 120 to the underside of support 110 (i.e., to the side that faces away from body portion 112 when leg segments 114 are expanded.) In the present embodiment, the attachment structures are loops formed onto an edge of each leg segment. Other attachment structures are possible, for example hooks or notches. In the present embodiment, patch 120 is stitched to each attachment structure. A series of additional stitches may be spaced along each leg to securely attach patch 120 to support 110. In another embodiment, the patch may be attached to the support using other means of attachment, for example adhesive bonding or thermal bonding.

Patch 120 comprises one or more materials that physically block and/or encourage growth of tissue to block an opening in the septal wall. Appropriate materials include, but are not limited to, a bioabsorbable compound, a polyester fabric, a polyurethane fabric, a polyethylene terephthalate fabric (e.g., Dacron®), a biocompatible woven fabric, collagen, another biologic, a material capable of promoting tissue growth, combinations thereof, and the like. Patch 120 must be flexible enough to be compressed into a delivery configuration when device 100 is contained within a delivery catheter and to expand into a deployment configuration when leg segments 114 self-expand radially outward from body segment 112. When in its delivery configuration, patch 120 is folded or otherwise compressed and at least partially contained within leg segments 114. When fully expanded into its deployment configuration, patch 120 is substantially flat for positioning against the septal defect. Patch 120 is shown as a round, flat structure in FIG. 1; however, one skilled in the art will appreciate that other shapes are possible and that the patch may be attached to some or all of the leg segments.

It is desirable that treatment device 100 be visible using intracardiac echocardiography (ICE), transesophogeal echocardiography (TEE), intravascular ultrasound, angioscopy, fluoroscopy, or another means of visualization to aid in positioning. Where fluoroscopy is utilized, any or all of treatment device 100 may be coated with a radiopaque material, or a radiopaque marker may be included on any portion of the device that would be useful to visualize.

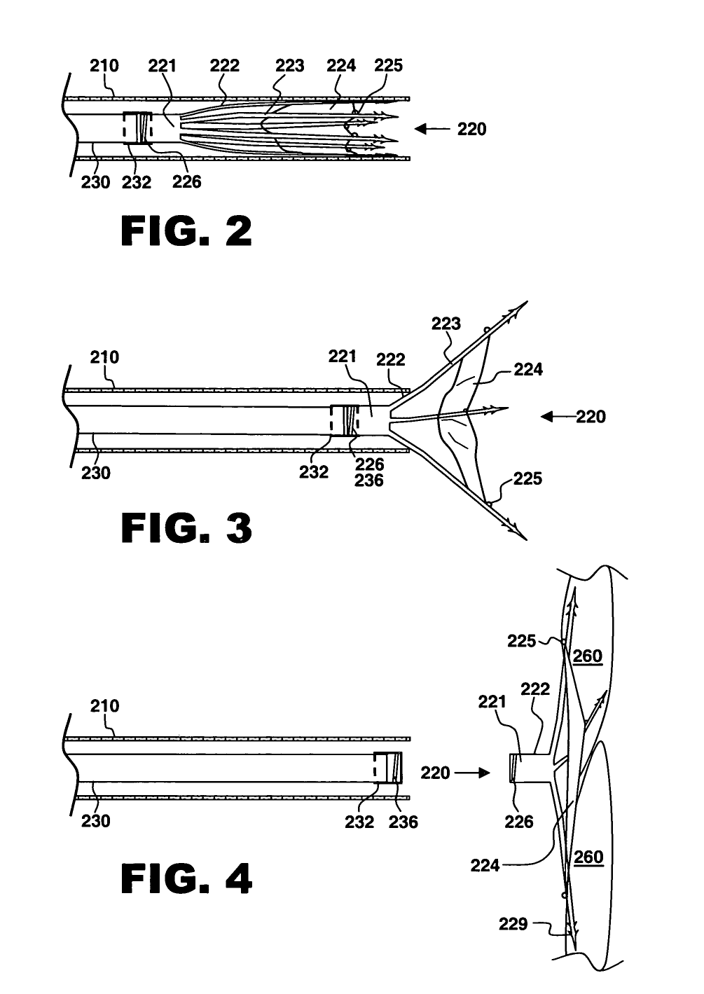

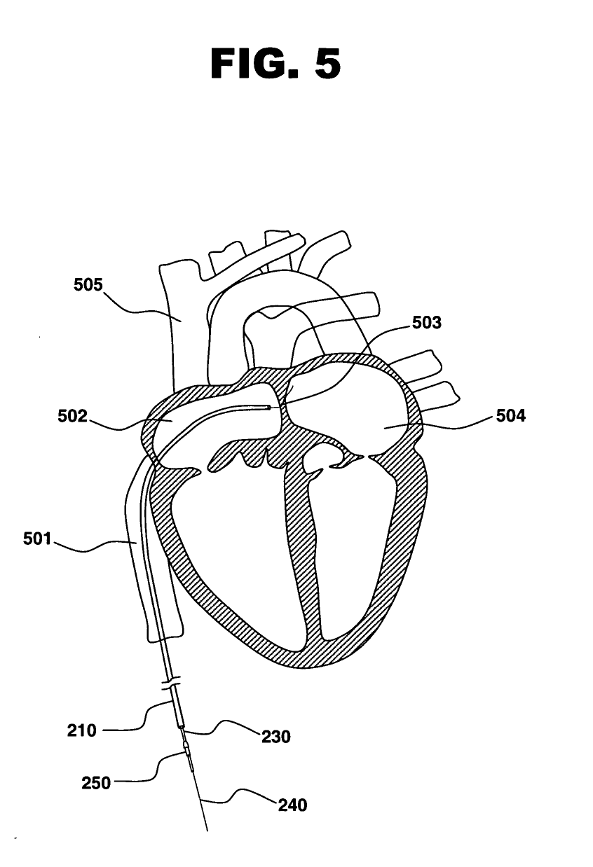

Another aspect according to the present invention is a system for treating a septal defect. One embodiment of the system, in accordance with the present invention, is illustrated in FIGS. 2-5, in which like elements share like reference numbers. The system comprises a delivery catheter 210, a treatment device 220, an elongated delivery device 230, and a guidewire 240. Treatment device 220 comprises a support 222 and a patch 224. Support 222 includes a body segment 221 and a plurality of leg segments 223. A connector 232 is attached to the distal end of delivery device 230. A releasable stop 250 is positioned on a proximal portion of delivery device 230. While described below and illustrated in FIG. 5 in the context of closing a patent foramen ovale (PFO), system 200 may be used to treat other septal defects.

Delivery catheter 210 is a conventional catheter, as is known in the art. Catheter 210 has an appropriate inner diameter to deliver treatment device 220 to a treatment site. The length of catheter 210 may depend upon the delivery route.

Treatment device 220 comprises support 222 and patch 224. In the present embodiment, support 222 is a section of nitinol tubing having evenly spaced longitudinal slots cut into an end portion of the tubing to form body segment 221 and six flexible leg segments 223, as shown in FIG. 2. FIGS. 3 and 4 show treatment device 220 in cross-section, with only three of the six leg segments depicted.

One skilled in the art will appreciate that the number of treatment device leg segments may be varied and that the support may be fabricated using other methods, including forming the support from one or more flat sheets of material. In addition, materials other than nitinol may be used, with at least leg segments 223 comprising a material capable of being preset into a desired shape. Such materials include, but are not limited to, a nickel-titanium alloy, a nickel-cobalt alloy, another cobalt alloy, a thermoset plastic, stainless steel, a stainless steel alloy, a biocompatible shape-memory material, a bioabsorbable shape-memory material, a biocompatible superelastic material, a bioabsorbable superelastic material, combinations thereof, and the like. Leg segments 223 are preset during manufacture into the radially expanded position they are to assume when deployed.

An antithrombotic component may be included in the chemical composition of a polymer used to form the device. Alternatively, a polymeric or metallic device may be coated with a polymer that releases an anticoagulant and thereby reduces the risk of thrombus formation. If desired, additional therapeutic agents or combinations of agents may be used, including antibiotics and anti-inflammatories.

Patch 224 comprises one or more materials that physically block and/or encourage growth of tissue to block an opening in the septal wall. Appropriate materials include, but are not limited to, a bioabsorbable compound, a polyester fabric, a polyurethane fabric, a polyethylene terephthalate fabric (e.g., Dacron®), a biocompatible woven fabric, collagen, another biologic, a material capable of promoting tissue growth, combinations thereof, and the like. Patch 224 must be flexible enough to assume both a compressed delivery configuration and an expanded deployment configuration. Each leg segment 223 may include an attachment structure 225 for attaching patch 224 to support 222.

Treatment device 220 is designed to be positioned using minimally invasive catheterization techniques. In FIG. 2, treatment device 220 is shown slidably received within the lumen of delivery catheter 210 for delivery to and deployment at a treatment area. Leg segments 223 are in a radially compressed, folded configuration that is maintained by the walls of the catheter lumen. Patch 224 is in its delivery configuration at least partially contained within leg segments 223.

As described more fully below, treatment device 220 is preferably deployed in two stages. As shown in FIG. 3, the device has achieved Stage 1, in which it is partially deployed (for example, about 60% to 80% deployed) and in the appropriate configuration to be placed in contact with and penetrate the tissue surrounding the septal defect.

In FIG. 4, the device has achieved Stage 2, in which it is fully deployed, with leg segments 223 self-expanded radially outward from body segment 221 into their preset shape. This radial expansion along with the force keeping the delivery catheter adjacent the septal defect implants a distal portion of each leg segment 223 in the tissue surrounding the septal defect, shown in FIG. 4 at 260. The leg segments are preferably implanted between the surfaces of the septal wall but may also pass through the tissue, piercing the septal wall. The leg segments may include one or more barbs 229 to increase the ability of leg segments 223 to grip tissue 260.

Patch 224 expands into its deployment configuration when the leg segments self-expand. When fully expanded into its deployment configuration, patch 224 is substantially flat for positioning against the septal defect, as seen in FIG. 4.

Treatment device 220 is deployed with the aid of elongated delivery device 230, which is slidably received within the lumen of catheter 210. In the present embodiment, delivery device 230 is a hypotube that is releasably attached to body segment 221 by means of connector 232. The outer surface of the treatment device body segment includes threads 226. Connector 232 is set onto the distal end of delivery device 230 and includes threads 236 on the inner surface of the connector that are complementary to the threads on body segment 221. Thus, treatment device 220 may be screwed onto delivery device 230 for delivery to and deployment at a treatment site and then unscrewed once the treatment device is fully deployed and ready to be released. A distal portion of the hypotube comprising delivery device 230 may include a spiral cut formed such that when delivery device 230 is rotated to disengage it from the fully deployed treatment device, the spiral tightens against itself rather than unwinding. The spiral cut increases flexibility of a distal portion of the delivery device without limiting transmission of torque to unscrew and release the treatment device.

One skilled in the art will appreciate that the treatment device body segment may have threads on an inside surface, and a connector having an outer diameter smaller than the inner diameter of the treatment device body may have threads on an outer surface. In another embodiment, the hypotube itself may include threads, eliminating the need for a connector. In yet another embodiment, neither the treatment device nor the delivery device may include threads, and the delivery device may be a length of hypotube that is not attached to treatment device 220. However, better control of delivery and deployment of the treatment device is possible if threads or other means of releasably attaching the treatment device to the delivery device are included.

Other delivery devices known in the art may be used. For example, in another embodiment, the delivery device may be biopsy forceps or another gripping device that holds the treatment device until it is properly positioned relative to the septal defect.

Stop 250 is shown in FIG. 5 attached to a proximal portion of delivery device 230. The stop is positioned to abut the proximal end of delivery catheter 210 when treatment device 220 has achieved Stage 1 deployment in which delivery device 230 has been slid in a distal direction within delivery catheter 210 until treatment device 220 is partially deployed and in the appropriate configuration to be placed in contact with and penetrate the tissue surrounding the septal defect. In the present embodiment, stop 250 is a wire torquing apparatus. Stop 250 is released to allow delivery device 230 to advance farther into delivery catheter 210 to achieve Stage 2, in which treatment device 220 is fully deployed, with leg segments 223 self-expanded radially outward from body segment 221 into their preset shape and with patch 224 expanded into its deployment configuration. A second stop may be placed to abut the proximal end of delivery catheter 210 when Stage 2 has been achieved.

In another embodiment, the stop(s) may be eliminated and an adaptor may be removably mounted on a proximal portion of the delivery catheter, the adaptor being movable between a first position in which the treatment device is fully within the lumen of the delivery catheter, a second position in which the treatment device is partially extended beyond a distal end of the delivery catheter, and a third position in which the treatment device is fully extended beyond the distal end of the delivery catheter. The adaptor may move amongst these positions using, for example, a ratcheting assembly or a thumb screw that contacts the delivery device to advance and retract it within the delivery catheter. In yet another embodiment, the body of the delivery device may simply include markings to indicate the Stage 1 and Stage 2 positions of the delivery device within the delivery catheter.

Delivery catheter 210 carrying treatment device 220 is passed through the venous system and into a patient's right atrium adjacent to the septal defect, in this embodiment a patent foramen ovale (PFO). Delivery may be accomplished as shown in FIG. 5, in which delivery catheter 210 has been inserted through the femoral vein into the common iliac vein, and through inferior vena cava 501 into right atrium 502, where it is positioned adjacent to foramen ovale 503. As illustrated, the treatment device is being delivered over guidewire 240, which is slidably received within the lumen of delivery catheter 210. In the present embodiment, treatment device body segment 231, set screw 232, and delivery device 220 all include lumens that can accommodate a guidewire having an outer diameter of, for example, 0.035 inch.

It is desirable that treatment device 220 be visible using intracardiac echocardiography (ICE), transesophogeal echocardiography (TEE), intravascular ultrasound, angioscopy, fluoroscopy, or another means of visualization to aid in positioning. Where fluoroscopy is utilized, any or all of treatment device 220 may be coated with a radiopaque material, or a radiopaque marker may be included on any portion of the device that would be useful to visualize.

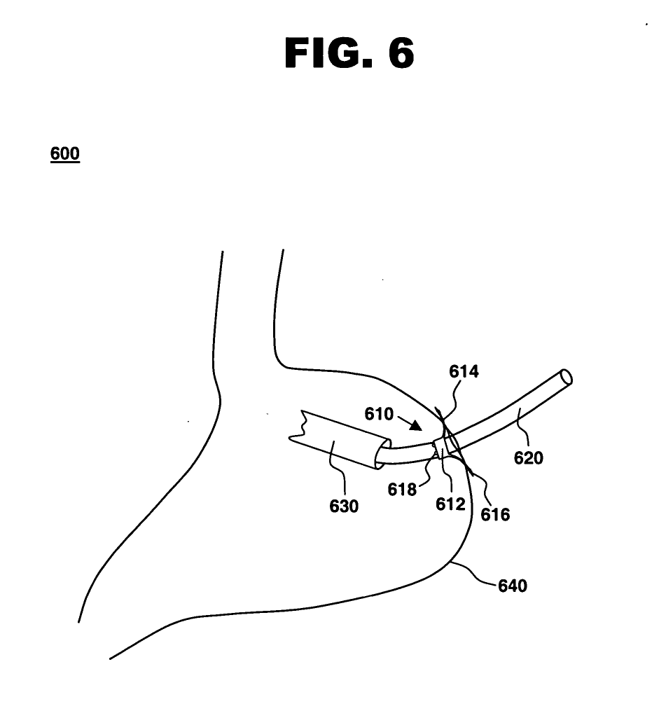

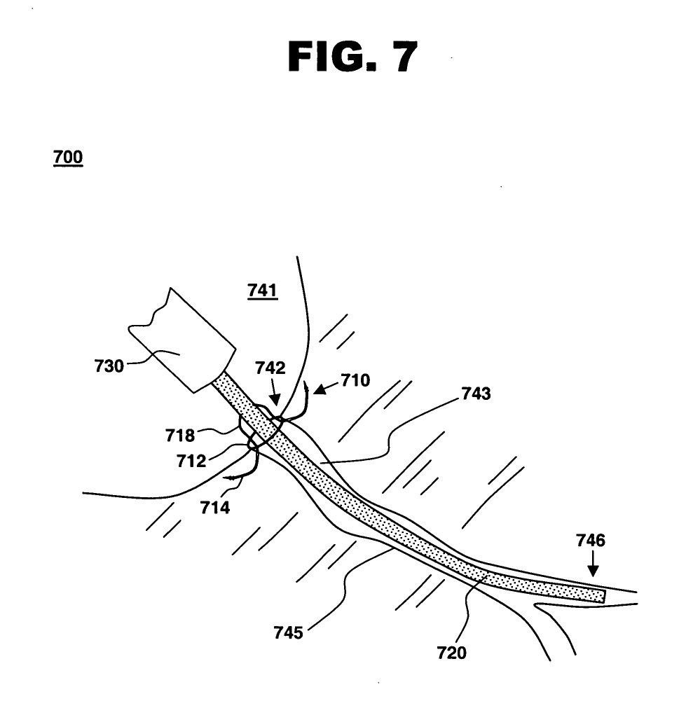

As will be apparent to one skilled in the art, the support shown at 110 in FIG. 1 may be readily adapted to anchor a medical component other than a patch. For example, a medical component may be carried within the body segment of the support rather than attached to the leg segments. FIGS. 6 and 7 illustrate two such systems.

The aspect according to the present invention illustrated in FIG. 6 at 600 is an indwelling medical system. System 600 comprises an anchor 610, an elongated member 620, and a retractable sheath 630. In the present embodiment, elongated member 620 is a gastrostomy tube, and retractable sheath 630 is a delivery catheter. FIG. 6 shows the system being deployed within stomach 640.

As illustrated in FIG. 6, anchor 610 includes a body segment 612 and a plurality of flexible leg segments 614. The leg segments may include barbs 616. During manufacture of anchor 610, leg segments 614 are bent outward and set at an angle of between 60 and 100 degrees from the longitudinal axis of body segment 612. The expanded device is threaded onto gastrostomy tube 620, with anchor 610 encircling a portion of the tube.

Attachment structures 618 are positioned on body segment 612, rather than being on the leg segments as in previously described embodiments according to the present invention. Anchor 610 is attached to the outer surface of gastrostomy tube 620 using sutures that connect the attachment structures to the outer surface of the tube. In another embodiment, the attachment structures may be eliminated, and anchor 610 may be bonded to the outer surface of the gastrostomy tube using, for example, a biocompatible adhesive such as polyethylene oxide.

Anchor 610 and gastrostomy tube 620 are delivered to the stomach within delivery catheter 630. Leg segments 614 assume a radially compressed, folded configuration within catheter 630, which encloses the leg segments during delivery. The system is passed through the mouth, down the esophagus, and into the inner lumen of the stomach. The system may track down a guidewire and may include a piercing catheter for piercing through the stomach wall and the abdominal wall to the outside of the body. Alternatively, the opening to the outside of the body may be made using a trocar or other sharp instrument prior to introducing system 600 into the stomach lumen.

Anchor 610 may be deployed using a two-stage process similar to that described above. Tube 620 holds the placed anchor 610 in position against the wall of stomach 640 while delivery catheter 630 is retracted to deploy the anchor. As anchor 610 is released from the catheter, leg segments 614 resume their preset shape, self-expanding radially outward from body segment 612, penetrating into the gastric muscle layer and anchoring gastrostomy tube 620.

FIG. 7 illustrates an alternative embodiment in which the elongated member is a pacemaker lead. FIG. 7 shows pacemaker lead 720 delivered through right atrium 741, coronary sinus ostium 742 and coronary sinus 743, and through great cardiac vein 745 to a target zone 746.

Anchor 710 encircles a portion of pacemaker lead 720 and is slidable along the lead until the anchor is fully deployed. The anchor includes a body segment 712 and a plurality of distal and proximal flexible leg segments, seen at 714 and 718, respectively. Proximal leg segments 718 serve as attachment members for fixing anchor 710 to lead 720 once the anchor is fully deployed.

Distal leg segments 714 are preset such that each of the leg segments is self-expanding radially outward at an angle of between 60 and 100 degrees from the longitudinal axis of body portion 712 when a retractable sheath, in this embodiment delivery catheter 730, is retracted. As shown in FIG. 7, body segment 712 of anchor 710 is a narrow band that serves as a pivot ring. As distal leg segments 714 self-expand radially outward, proximal leg segments 718 pivot inward and engage lead 720.

Pacemaker lead 720 is delivered to its target position using techniques known in the art. Once the distal end of lead 720 has been conventionally attached within target zone 746, anchor 710 is positioned adjacent to coronary sinus ostium 742 and deployed using a two-stage process such as has been described above. An inner catheter may be used to hold anchor 710 in position as delivery catheter 730 is withdrawn to deploy the anchor. Leg segments 714 are implanted in tissue surrounding the coronary sinus ostium, anchoring the lead in the vein and reducing the risk of the lead being displaced over time.

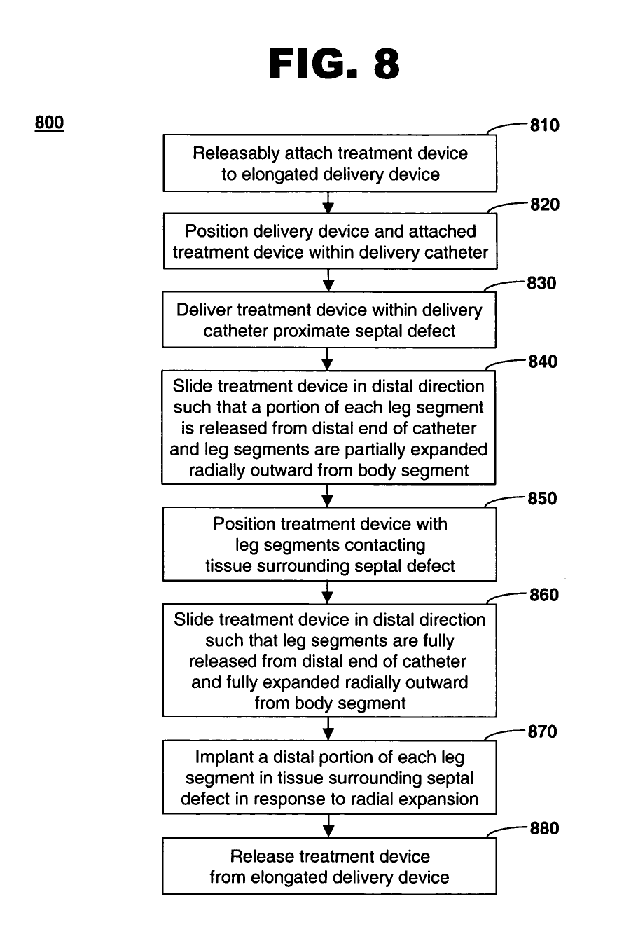

Still another aspect according to the present invention is a method of treating a septal defect. FIG. 8 shows a flow diagram of one embodiment of the method in accordance with the present invention.

A treatment device is releasably attached to an elongated delivery device (Block 810). The treatment device comprises a support and a patch attached to the support. The support includes a body segment and a plurality of flexible leg segments. Treatment devices in accordance with the present invention are shown in FIGS. 1-4. The delivery device may be as described above and illustrated in FIGS. 2-4. In the present embodiment, the treatment device is releasably attached to the delivery device by means of threading on both the treatment device and the delivery device that allows the treatment device to be screwed onto the delivery device.

The delivery device and attached treatment device are positioned within a delivery catheter (Block 820). This may be accomplished by inserting the proximal end of the delivery device into the distal end of the delivery catheter, and feeding the delivery device and attached treatment device through the delivery catheter until the treatment device is drawn into the distal end of the delivery catheter as shown in FIG. 2. As the leg segments of the treatment device enter the catheter, they are folded toward each other, becoming radially compressed. The attached patch is thereby compressed into a delivery configuration at least partially contained within the leg segments.

The treatment device is deployed in two stages. At Stage 1 deployment, shown in FIG. 3, the treatment device is partially deployed (for example, about 60% to 80% deployed) and in the appropriate configuration to be placed in contact with and penetrate the tissue surrounding the septal defect. At Stage 2 deployment, shown in FIG. 4, the treatment device is fully deployed, with the leg segments self-expanded radially outward from the body segment and the patch fully expanded into its deployment configuration.

One or more stops may be attached to a proximal portion of the delivery device to indicate when a deployment stage has been achieved. For example, the treatment device may be drawn into the catheter until it assumes the Stage 1 configuration. To mark this stage, a releasable stop, for example a wire torquing apparatus, may be attached to the delivery device and positioned abutting the proximal end of the catheter. The treatment device may then be drawn fully within the catheter, with the releasable stop drawn proximal to the end of the catheter and in position to indicate to the medical professional when Stage 1 deployment has been achieved. If desired, a stop indicating Stage 2 deployment may be similarly attached prior to attaching the Stage 1 deployment indicator.

It will be apparent to one skilled in the art that the stop(s) may be eliminated, and other means for achieving staged deployment may be employed. For example, the body of the delivery device may include markings that indicate the position of the delivery device within the delivery catheter at Stage 1 and Stage 2 deployment. Alternatively, an adaptor may be removably mounted on a proximal portion of the delivery catheter, the adaptor being movable between a first position in which the treatment device is fully within the lumen of the delivery catheter, a second position in which the treatment device is partially extended beyond a distal end of the delivery catheter (Stage 1 deployment), and a third position in which the treatment device is fully extended beyond the distal end of the delivery catheter (Stage 2 deployment). The adaptor may move amongst these positions using, for example, an assembly that contacts the delivery device to advance and retract it within the delivery catheter.

In another embodiment, the delivery device may be supplied already threaded into the delivery catheter, and the treatment device may be attached to the distal end of the delivery device and drawn into the delivery catheter as described above. In yet another embodiment, both the delivery device and the treatment device may be supplied already positioned within the delivery catheter.

The treatment device, fully contained within the lumen of the catheter, is delivered proximate a septal defect (Block 830). One path for delivering the treatment device is shown in FIG. 5. In the present embodiment, the treatment device is delivered over a guidewire that has been previously introduced using, for example, a Brockenbrough curved needle. When closing a patent foramen ovale, the guidewire catheter is inserted through the femoral vein into the common iliac vein, through inferior vena cava 501 into right atrium 502, and passed through foramen ovale 503 into left atrium 504. Other paths are available, including through the radial vein into the brachial vein, through the subclavian vein, and through superior vena cava 505 into right atrium 502. The guidewire catheter is then removed, leaving the guidewire in place to guide the treatment device into position over the right atrial fossa of the foramen ovale.

Once the treatment device is in place proximate the septal defect, the treatment device is slid in a distal direction such that a portion of each leg segment is released from the distal end of the catheter and the leg segments are partially expanded radially outward from the body segment (Block 840). The treatment device is positioned with the leg segments contacting tissue surrounding the septal defect (Block 850). The treatment device is slid farther in a distal direction until the leg segments are fully released from the distal end of the catheter and fully expanded radially outward from the body segment (Block 860). In response to the full radial expansion of the leg segments, a distal portion of each leg segment is implanted in tissue surrounding the septal defect, thereby positioning the patch against the septal defect (Block 870). The leg segments are preferably embedded within the septal wall (i.e., implanted between the surfaces of the tissue) but may also pass through the wall.

Once the leg segment distal portions have been implanted in the tissue, the treatment device is released from the delivery device (Block 880). In the present embodiment, this is accomplished by unscrewing the delivery device from the treatment device. The delivery catheter and delivery device may then be removed from the patient, leaving the treatment device in place with the patch positioned against the septal defect. The patch comprises one or more materials that physically block and/or encourage growth of tissue to block an opening in the septal wall, thereby treating a patent foramen ovale or other septal defect.

While specific embodiments have been disclosed, various changes and modifications can be made without departing from the spirit and scope of the invention. The scope of the invention is indicated in the appended claims, and all changes and modifications that come within the meaning and range of equivalents are intended to be embraced therein.

What is claimed is:

1. A device for treating a septal defect, comprising:
a support including a body segment and a plurality of flexible leg segments, wherein the leg segments self-expand radially outward from the body segment as the support is released from a delivery catheter; and
a patch attached to the support.

2. The device of claim 1 wherein the patch expands from a delivery configuration to a deployment configuration when the leg segments self-expand radially outward from the body segment.

3. The device of claim 1 wherein each leg segment includes at least one patch attachment structure.

4. The device of claim 3 wherein the patch attachment structure comprises a loop.

5. The device of claim 1 wherein each leg segment includes at least one barb.

6. The device of claim 1 wherein at least the leg segments comprise a material selected from a group consisting of a nickel-titanium alloy, a nickel-cobalt alloy, a cobalt alloy, a thermoset plastic, stainless steel, a stainless steel alloy, a biocompatible shape-memory material, a bioabsorbable shape-memory material, a biocompatible superelastic material, a bioabsorbable superelastic material, and a combination thereof.

7. The device of claim 1 wherein the patch comprises a material selected from a group consisting of a bioabsorbable compound, a polyester fabric, a polyurethane fabric, a polyethylene terephthalate fabric, a biocompatible woven fabric, collagen, a biologic, a material capable of promoting tissue growth, and a combination thereof.

8. The device of claim 1 wherein a portion of each leg segment implants between the surfaces of a septal wall as the support is released from a delivery catheter.

9. A system for treating a septal defect, comprising:
a delivery catheter; and
a treatment device slidably received within a lumen of the delivery catheter, the treatment device having a support and a patch attached to the support, the support including a body segment and a plurality of flexible leg segments, wherein the leg segments self-expand radially outward from the body segment as the device is released from the delivery catheter.

10. The system of claim 9 wherein the patch expands from a delivery configuration to a deployment configuration when the leg segments self-expand radially outward from the body segment.

11. The system of claim 9 wherein a portion of each leg segment implants between the surfaces of a septal wall as the treatment device is released from the delivery catheter.

12. The system of claim 9 further comprising:
an elongated delivery device slidably received within the lumen of the delivery catheter.

13. The system of claim 12 wherein the delivery device is releasably attached to the body segment of the treatment device.

14. The system of claim 12 wherein the body segment of the treatment device includes threads.

15. The system of claim 14 wherein the delivery device is a hypotube and wherein a distal portion of the hypotube includes threads complementary to the treatment device threads.

16. The system of claim 14 further comprising:
a connector attached to a distal end of the delivery device, wherein the connector includes threads complementary to the treatment device threads.

17. The system of claim 9 further comprising:
a guidewire slidably received within the lumen of the delivery catheter.

18. The system of claim 9 further comprising:
at least one releasable stop positioned on a proximal portion of the elongated delivery device.

19. The system of claim 9 wherein the treatment device is deployed in two stages.

20. An indwelling medical system, comprising:
an elongated member;
an anchor encircling a portion of the elongated member, the anchor including a body segment and a plurality of flexible leg segments; and
a retractable sheath enclosing the flexible leg segments, wherein at least a portion of the leg segments self-expand radially outward from the body segment when the sheath is retracted.

21. The system of claim 20 wherein at least a portion of the flexible leg segments pivot inward to engage the elongated member when the sheath is retracted.

22. A method of treating a septal defect, comprising:
delivering a treatment device in a lumen of a catheter proximate a septal defect, the treatment device comprising a support and a patch attached to the support, the support including a body segment and a plurality of flexible leg segments;
sliding the treatment device in a distal direction such that a portion of each leg segment is released from a distal end of the catheter and the leg segments are partially expanded radially outward from the body segment;
positioning the treatment device such that the leg segments contact tissue surrounding the septal defect; and
sliding the treatment device in a distal direction such that each leg segment is fully released from the distal end of the catheter and the leg segments are fully expanded radially outward from the body segment; and
implanting a distal portion of each leg segment in the tissue surrounding the septal defect in response to the full radial expansion of the leg segments, thereby positioning the patch against the septal defect.

23. The method of claim 22 wherein delivering a treatment device in a lumen of a catheter proximate a septal defect comprises passing a guidewire through the septal defect and delivering the treatment device over the guidewire.

24. The method of claim 22 further comprising:
prior to delivering the treatment device in a lumen of a catheter proximate a septal defect, releasably attaching the treatment device to an elongated delivery device; and
positioning the treatment device and elongated delivery device within the delivery catheter.

25. The method of claim 24 further comprising:
after implanting a distal portion of each leg segment in the tissue, releasing the treatment device from the elongated delivery device.

26. The method of claim 22 wherein implanting a distal portion of each leg segment in the tissue comprises implanting a distal portion of each leg segment between the surfaces of the tissue.
